(19) United States
(12) Patent Application Publication
(10) Pub. No.: US 2024/0042090 A1
(43) Pub. Date: Feb. 8, 2024

(54) SYSTEM AND METHOD FOR AIR STERILISATION

(71) Applicant: MEDAIR LIMITED, Wan Chai (HK)

(72) Inventors: Yuen Yee CHENG, Riverwood (AU); Andreas SUHRBIER, Bunya (AU); Hua FAN, Randwick (AU)

(21) Appl. No.: 18/269,107

(22) PCT Filed: Dec. 22, 2021
(86) PCT No.: PCT/AU2021/051540

§ 371 Date: Jun. 22, 2023

(30) Foreign Application Priority Data
Dec. 24, 2020 (AU) 2020904854

(51) Int. Cl. A61L 9/22; A61L 9/01; H01T 19/04

(52) U.S. Cl. CPC A61L 9/22; A61L 9/01; H01T 19/04; A61L 2209/14

(57) ABSTRACT

An air purification system has an air inlet far and an air outlet, with a conduit between the air inlet and the air outlet. Disposed within the conduit in the direction of air flow, there is included at least one ionizer creating a first air ionisation zone within which, in use, aerosols and/or airborne particles in incoming air are ionised become charged. In the conduit there is further a charge reduction stage creating a first charge reduction zone that includes a charge reduction device located across a flow path of the airflow, terminating the air ionisation zone, such that the wherein as charged aerosols and/or particles pass therethrough, and in passing through the charge reduction stage the charged aerosols and/or particles are attracted to the charge reduction stage and on contact with the charge reduction stage become at least partially discharged.

### TECHNICAL FIELD

This disclosure relates to air purification, by sterilisation, removal, or both, of airborne particles and/or aerosols using ionisation of air. A particular application is in the sterilisation and/or removal of airborne pathogens, aerosols containing pathogens, or both.

### BACKGROUND ART

Presently, systems exist which utilise negative ions for the purpose of air purification. Some such systems work mainly to increase the concentration of negative ions in the air. However, simply increasing the concentration of negative ions does not improve the adsorption or removal of impurities or other particles in the air, such as aerosols, aerosols containing infectious agents, particulate matter, viruses, bacteria and the like.

It is to be understood that, if any prior art is referred to herein, such reference does not constitute an admission that the prior art forms a part of the common general knowledge in the art, in Australia or any other country.

### SUMMARY

In one aspect, herein disclosed is an air purification system, comprising: an air inlet for accepting an airflow into the system and an air outlet for the airflow to exit the system, with a conduit between the air inlet and the air outlet. Disposed within the conduit in the direction of air flow, there are included in the system: an ionizer creating a first air ionisation zone within which, in use, aerosols and/or airborne particles in incoming air are ionised become charged; and a charge reduction stage creating a first charge reduction zone. As charged aerosols and/or particles pass therethrough, the charged aerosols and/or particles are attracted to the charge reduction stage and on contact with the charge reduction stage become at least partially discharged, the charging and subsequent discharging removing, inactivating and/or eliminating at least some aerosols and/airborne particles. The charge reduction stage comprises a charge reduction device which is located in or across a flow path of the airflow. The flow path is in a flow channel defined by the housing.

In some forms, the ionizer comprises a plurality of electrodes distributed within the first ionization zone and charged, in use, to generate a negative ion discharge so as to ionise the aerosols and/or airborne particles in incoming air passing through the first air ionisation zone.

In some forms, the electrodes are charged to provide a corona discharge.

In some forms, the electrodes are plated with silver, gold, copper, or platinum.

In some forms, adjacent electrodes are separated by a distance of at least 0.5 cm.

In some forms, adjacent electrodes are separated by a distance of at least 1.5 cm.

In some forms, the ionizer comprises a frame onto which the electrodes are mounted, the frame being generally positioned across the flow path.

In some forms, the electrodes are evenly spaced across the frame.

The system may comprise one or more further ionisation zones.

In some forms, at least two of the two or more ionisation zones are configured to charge the aerosols and/or airborne particles in the incoming air to different electrical potentials.

In some forms, the ionisation zone which is closest to the inlet is configured to charge the aerosols and/or airborne particles in the incoming air to provide a least ionisation level compared to other ionisation zone(s).

The system may comprise one or more further charge reduction stages.

In some forms, at least two of the charge reduction stages and two of the ionisation zones in the conduit are provided in alternating fashion.

The charge reduction device may be grounded or connected to a voltage source.

In some forms, the charge reduction device comprises a structure having a textured surface.

In some forms, a material of the charge reduction device comprises an anti-bacterial or sterilising additive.

In some forms, the charge reduction device includes at least one mesh layer. The mesh layer may be a stainless steel mesh layer. The mesh size may be around 50 to 60 apertures per squared centimetre.

The system may comprise at least two charge reduction devices arranged in series in the conduit, the two charge reduction devices having different electrical potentials.

The system may comprise at least an exit ionisation zone which is provided adjacent the outlet.

The system may have mounted therein one or more light sources.

The system may comprise an air pressure generation device to drive the airflow.

The system may comprise an air redirection device configured to disturb the airflow as it travels from the ionizer to the charge reduction stage. In some forms, the disturbed airflow comprises flows in different directions. In some forms, the air redirection device is configured to define one or more airflow path therein which are longer than a length between the ionizer and the charge reduction stage. In some forms, the air redirection device comprises a Tesla air flow arrangement.

The system may comprise a filtration device located adjacent the ionizer. The filtration device may comprise a material which absorbs ozone molecules. The material may be activated carbon. In some forms, the filtration device comprises a layered structure. The layered structure may includes outer layer which are fabric layers comprising silver fibres.

The system may be retrofitted in an existing building, transport or vehicular air circulation system.

In a second aspect, herein disclosed is a method of air purification, including: creating an air pressure to draw an airflow through a flow path; providing positive or negative ions in the flow path, so as to ionise aerosols and/or airborne particles in the flow path; and altering an electrical potential of at least some of the ionised aerosols and/or airborne particles to inactivate those ionized aerosols and/or airborne particles, by placing in the flow path a charge reduction device which will contact said at least some of the ionised aerosols, airborne particles, or both, the charge reduction device being adapted so that the airflow will pass therethrough.

Another aspect provides an purification system, comprising: an air inlet for accepting an airflow into the system and an air outlet for the airflow to exit the system, with a conduit between the air inlet and the air outlet, disposed within the conduit in the direction of air flow including: at least one ionizer creating an air ionisation zone within which, in use, aerosols and/or airborne particles in incoming air are ionised become charged, and a charge reduction stage creating a first charge reduction zone, the charge reduction stage comprising a charge reduction device which is located across a flow path of the airflow, terminating the air ionisation zone, such that the charged aerosols and/or particles pass therethrough, and in passing through the charge reduction stage the charged aerosols and/or particles are attracted to the charge reduction stage and on contact with the charge reduction stage become at least partially discharged, the action of charging and subsequent discharging biologically inactivating at least some aerosols and airborne particles.

Anther aspect provides a method of air purification, including: creating an air pressure to draw an airflow through a flow path; providing positive or negative ions in the flow path, so as to ionise aerosols, airborne particles, or both in the flow path; and altering an electrical potential of at least some of the ionised aerosols, airborne particles, or both, to biologically inactivate said at least some of the ionized aerosols and/or airborne particles, by placing in the flow path a charge reduction device comprising a charge reduction device which is located across a flow path of the airflow such that the airflow will pass therethrough with charged aerosols and/or particles passing therethrough after being ionised, and said at least some of the ionised aerosols, airborne particles, or both, will contact the charge reduction device and as a result of contact alter electrical potential to thereby biologically inactivating the ionised aerosols, airborne particles or both.

The charge reduction device may have a surface which is connected to electrical ground or which has applied thereto an electrical potential which is set at a level to cause said altering of the electrical potential of at least some of the ionised aerosols and/or airborne particles.

The method may include further ionising the particles after said altering of the electrical potential of at least some of the ionised aerosols and/or airborne particles.

### BRIEF DESCRIPTION OF THE DRAWINGS

Embodiments will now be described by way of example only, with reference to the accompanying drawings in which

FIG. 1 is a schematic drawing showing an air purifying device in accordance with an embodiment of the present invention;

FIG. 2 is a schematic drawing showing an air purifying device in accordance with another embodiment of the present invention;

FIG. 3 is a schematic drawing showing an air purifying device in accordance with a further embodiment of the present invention;

FIGS. 4 to 9 depict examples of different configurations of ionisation zones and charge neutralisation zones;

FIG. 10 is a schematic depiction of the embodiment shown in FIG. 2, together with a control and power arrangement;

FIG. 11 (a) is a picture of an experimental set up to test the effectiveness of the air purification device;

FIG. 11 (b) is a picture of the nebulizer used in FIG. 11(a);

FIG. 12 (a) is a table showing a component switching sequence in a prototype experiment using SARS-CoV-2 aerosol;

FIG. 12 (b) is a photograph of the 96 well plate after a test run with the negative electrodes off, in which 85 out of 96 wells captured infectious aerosols as shown by viral cytopathic effects (CPE) indicated by reduced crystal violet staining;

FIG. 12 (c) is a photograph of the 96 well plate after a test run with the negative electrodes on, in which 0 out of 96 wells captured infectious aerosols as shown by viral cytopathic effects (CPE) indicated by reduced crystal violet staining, indicating a 100% removal of infectious aerosol compared with FIG. 12(b);

FIG. 13 is a table showing a component switching sequence in a prototype experiment using a Getah virus aerosol;

FIG. 14(a) is a photograph of the 96 well plate after a test run with the negative electrodes off, in which 90 out of 96 wells captured infectious aerosols as shown by viral cytopathic effects (CPE) indicated by reduced crystal violet staining;

FIG. 14(b) is a photograph of the 96 well plate after a test run with the negative electrodes off, in which 0 out of 96 wells captured infectious aerosols as shown by viral cytopathic effects (CPE) indicated by reduced crystal violet staining;

FIG. 15 is a schematic depicting an air purification system installed within an existing system in which there is an airflow;

FIG. 16 is a schematic depiction of an embodiment of a filtration device;

FIG. 17 is a schematic depiction of an embodiment of an air purification system which includes therein an air redirection device for promoting the mixing of air and ions;

FIG. 18 is a partial cut-away view of an air purification device in accordance with an embodiment of the present invention;

FIG. 19-1 is a perspective view of an ionisation device and a charge reduction device in accordance with an embodiment of the present invention;

FIG. 19-2 is a perspective view of an ionisation device and a charge reduction device in accordance with another embodiment of the present invention;

FIG. 19-3 is a perspective view of a charge reduction device having a planar configuration.

### DETAILED DESCRIPTION

In the following detailed description, reference is made to accompanying drawings which form a part of the detailed description. The illustrative embodiments described in the detailed description, depicted in the drawings, are not intended to be limiting. Other embodiments may be utilised and other changes may be made without departing from the spirit or scope of the subject matter presented. It will be readily understood that the aspects of the present disclosure, as generally described herein and illustrated in the drawings can be arranged, substituted, combined, separated and designed in a wide variety of different configurations, all of which are contemplated in this disclosure.

Currently there is increasing evidence suggesting that aerosols do present an infection risk, especially in settings with high aerosol generating procedures (for example, such as some ear, nose and throat procedures) or during high aerosol generating activities (for example, coughing, sneezing, strenuous exercise, shouting, singing), with even the wearing of N95 masks not completely blocking stimulated transmission. In specific settings aerosol transmission might be viewed as more likely, in particular closed spaces with poor ventilation e.g. certain hospital settings, aeroplanes etc. Air purification systems that could remove infectious aerosols could potentially make a major contribution to reducing SARS-CoV-2 transmission risk in certain settings.

Herein described is a system and method for air purification, making use of the ionisation of air to remove, eliminate, or inactive infectious aerosols or airborne particles or pollutants, in particular airborne organic pollutants such as airborne pathogens, or a combination. The system will draw in ambient air for purification and then blow out processed air. It may be a stand-alone unit. However, the same concept may also be incorporated into another system in which there is an airflow or where an air flow is expected to be generated, such as an air condition system or an air filtering or circulation system. These other systems can themselves be stand-alone units, or they may be integrated within a larger structure such as a building, a plane, etc. They may alternatively be part of a wearable apparatus, such as a personal protection equipment. Potential applications can be found in e.g., domestic, commercial, or clinical settings. There may also be particular application in transportation systems, particularly those where passengers are confined in enclosed spaces during travel.

In further embodiments, the concept may be incorporated into a wearable device such as a personal protection equipment (PPE), for the generation of “clean” air flow—i.e., air flow from which infectious aerosols, or other contaminants, or both, are at least partially removed, eliminated, or inactivated—to supply to the PPE wearer.

In the below, where ever used, the word “particles” is considered to generally encompass various substances that are airborne in the airflow such as particles of viruses or bacteria, and other organic or inorganic contaminants.

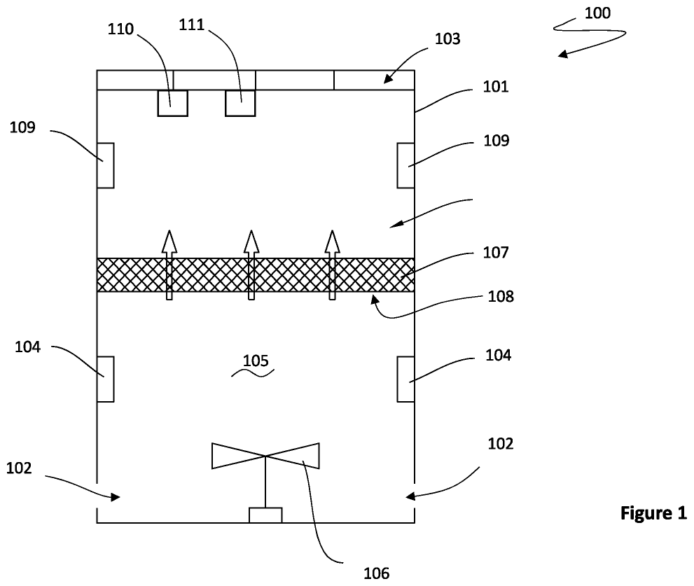

FIG. 1 depicts an air purification system 100 in accordance with one embodiment. The system 100 includes a housing 101 which provides the structure in which a number of components are mounted. The housing 101 is configured to provide one or more air inlets 102 and through which air is drawn into the housing 101, and one or more air outlets 103 through which air will exit the housing 101. An airflow path is thereby defined within the housing, to guide the flow of air from the inlet(s) 102 to the outlet(s) 103. In FIG. 1, a fan 106 is provided to provide the negative pressure for drawing in air. Here, the fan 106 is shown to include an axial flow impeller installed in the housing 101. This arrangement may help to reduce the size of the housing required.

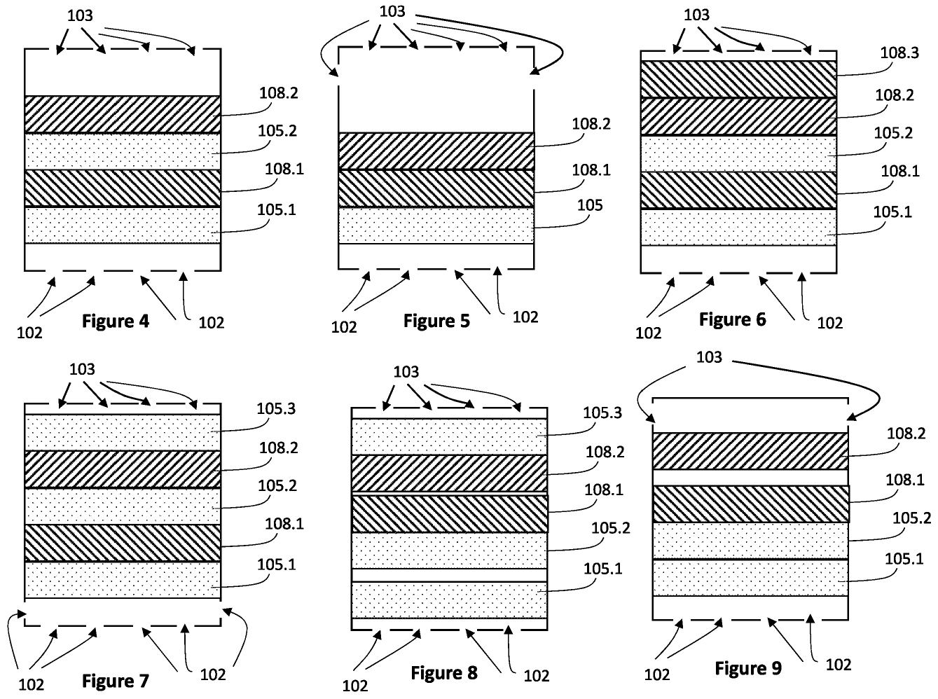

It will be appreciated that in other embodiments including fans, the fans need not be located adjacent the outlet as shown in FIG. 1, as long they provide the required pressure to create or facilitate the airflow. Also, the inlet(s) 102 do not need to be provided on the side wall of the housing 101, the sidewall being adjacent the airflow path, as shown in FIG. 1. The sidewall can further considered to define a flow channel in which the air flows. The inlet(s) 102 may instead be provided on an end wall (e.g., see FIGS. 4 to 8), which is in the airflow path. Similar, the outlet(s) 103 may be provided on the sidewall of the housing rather than an end wall as shown in FIG. 1.

Adjacent the inlet(s) 102 there are provided a plurality of electrodes 104 which in use will be negatively charged by electrical connection to a negative voltage module (not shown) which is configured to provide the negative charge to the electrodes 104. The power module (not shown) for energising the electrodes 104 module can be located outside the housing 101. It may be controllable to adjust the voltage level.

The electrodes 104 are configured to generate a corona discharge so as to create negative ions. Incoming aerosols and/or particles that enter the housing 101 with the airflow, which are initially electrically neutral, will be attracted to the negative ions and once in contact, charged and thus ionised by the negative ions. The space within the housing 101 in which the negative ions is generated to ionise the aerosols and/or particles may thus also be referred to as the ionisation chamber or ionisation zone 105. The electrodes 104 provided for ionising the aerosols and/or particles in the ionisation chamber 105 may also be referred to as the ionisation electrodes 104.

The ionisation electrodes 104 may be evenly positioned about the housing wall in the ionisation chamber 105, but this is not strictly required. It is also not strictly required that they be provided in aligned pairs as illustrated in FIG. 1. Adjacent pairs of electrodes will preferably have sufficient separation from each other so as to minimise ozone production.

The voltage of the voltage source should be chosen such that it is high enough to generate a sufficient concentration of negative ions within the ionising chamber 105 so as to ionise most of the incoming aerosols and/or particles, but still at a level such that ozone generation is minimised.

A charge reduction stage 107 is located between the plurality of electrodes 104 and the outlet(s) 103, and thus between the ionisation chamber 105 and the outlet(s) 103. The ionisation chamber 105 can therefore also be considered to terminate at a charge reduction zone 108 in which the charge reduction stage 107 is located. It would be appreciated that depending on the amount of charge reduction achieved by the charge reduction stage 107, the charges of the ionised aerosols and/or particles could be neutralised. Thus the charge reduction stage 107 could also provide charge neutralisation in some cases.

The charge reduction or neutralisation stage 107 will include a device formed of a conductive material, and it is therefore a conductive device. The purpose of the charge reduction or neutralisation device 107 is to reduce or neutralise the charges in those ionised aerosols and/or particles such as but not necessarily pathogens, that come into contact with the charge reduction or neutralisation stage 107 as they move with the airflow. In doing so, the charge reduction or neutralisation stage 107 causes a change in the electrical potential of at least some of the ionised aerosols and/or particles, which will be attracted by an electrical force to the charge reduction stage 107, and so removed from the airflow. Depending on the change, the change in the electrical potential may result in a temporary flow of electrons to or from the aerosols and/or particles, to reduce the electrical potential of, or neutralise, the aerosols and/or particles.

There may be an instantaneous, sudden, or rapid change in the electrical potential level of the ionised aerosols and/or particles (which have come into contact with the charge reduction stage 107), and hence an instantaneous, sudden, or rapid, temporary electron discharge from or influx into the aerosols and/or particles. This discharge or influx is expected to continue until the aerosols and/or particles are neutralised, or until the aerosols and/or particles physically detach from the charge reduction stage 107.

In most embodiments, the charge reduction or neutralisation stage 107 will provided a grounded device. However, the charge reduction or neutralisation stage 107 may provide a device which is charged to a non-zero electrical potential.

More generally, the charge reduction or neutralisation device 107 will be at a voltage (zero, positive, or negative) which will cause a reduction in the level of ionisation in the ionised aerosols and/or particles. For instance, when the aerosols and/or particles are negatively ionised, the electrical potential of the charge reduction or neutralisation stage 107 is less negative (i.e., more positive) compared to the ionised aerosols and/or particles. The difference in potentials aims to stimulate rapid, near instantaneous discharge which has an effect of biologically inactivating or eliminating or removing infectious aerosols or pathogens. Similarly, where the aerosols and/or particles are ionised with positive charges, the charge reduction or neutralization device 107 may be set at a potential which will reduce the positive charges and also stimulate the aforementioned discharge or influx to inactivate, eliminate or remove the pathogen.

The charge reduction or neutralisation stage 107 is configured to provide holes or passages therethrough to allow the passage of air. The difference in the electrical charge between the ionised aerosols and/or particles (substances such as pathogens, dust, etc) and the surface of the charge reduction device 107 causes the charged (ionised) aerosols and/or particles to be attracted to the device surface and discharge. The charge reduction, possibly neutralisation, also has the benefit of reducing release of ions from the device.

The following example uses negative ions, negatively charging incoming aerosols or airborne particles. For convenience the charge reduction or neutralisation stage 107 will be referred to as a charge reduction stage 107. Surfaces of the charge reduction stage 107 in the airflow path may be bombarded by the ionised aerosols and/or particles. The surfaces, by their relatively positive electrical charge, attract the more negatively charged aerosols and/or particles. Thus, the charge reduction stage 107 will be configured to present a large enough surface area so as to “neutralise” (or make less ionised) as much of the charged aerosols or airborne particles as possible, preferably without impeding the air flow or without causing a substantial impedance to the airflow.

To increase the surface area available for contact, the surfaces presented by the charge reduction stage 107 may be textured, such as being a rough rather than smooth surface. The surface may be provided with bumps, ridges, grooves, or the like. It will be appreciated that as long as the above general requirements are met, the exact geometry or configuration of the charge neutralisation device is not critical. The charge reduction stage 107 may include one or more substantially planar devices such as a mesh which extends in the plane across the cross section of the flow path. It may instead include one or more non-planar devices, each having a non-negligible or a more substantial thickness in the direction of the flow path. It may have a regular or irregular shape. For example, fibrous or webbed structures, such as steel wool or a sponge like device may be used. Further examples include other three-dimensional structures such as a cage or a three-dimensional matrix. A combination of the aforementioned configurations may be used.

Any conductive material may be used, for example conductive metals, conductive polymers etc. Materials may also be chosen for additional antibacterial, antimicrobial or antiviral properties, for example silver or copper or alloys thereof, or materials may be coated, plated or doped with materials having sanitizing properties.

As the aerosols and/or airborne particles are drawn into the housing 101, they are ionised in the ionisation zone 105. The ionisation can be provided using any known ionization technology. For example, currently commercially available ionizers utilize corona discharge from electrodes to ionise molecules in the air. Various electrode configurations are known to be able to provide corona discharge and in embodiments of the present invention the voltage is controlled to cause ionisation with minimal or no ozone production.

The now ionised aerosols and/or particles will continue to be drawn by the air pressure toward the outlet(s) 103, thereby entering the charge reduction zone 108 where they may come into contact with the surfaces of the charge reduction stage 107. Upon this contact, due to the electrical differential between the charged aerosols and/or particles and the surface of the charge reduction stage 107, an electron discharge may flow from the charged aerosols and/or particles (if negatively ionised) toward the surface of the charge reduction stage 107. The resulting temporary current flow alters the electrical potential of the charged aerosols and/or particles. The resulting temporary current flow also works to biologically inactivate or eliminate aerosols and/or particles such as airborne pathogens. The electrical attraction of the charged aerosols and/or particles to the charge reduction stage 107 may also remove at least some of the charged substances such as aerosols or particles from the airflow.

Device prototype had been tested in an independent PC3 laboratory for SARS-CoV-2 and PC2 laboratory for Getah virus; two different viruses from different genera. In a prototype testing done, an experiment demonstrated a 100% reduction of infectious coronavirus (SARS-COV-2) aerosols, and another experiment demonstrated a 100% reduction of infectious Getah virus aerosols. The experimentation and result will be disclosed in more detail later in this document.

However, it would be expected that the disclosed embodiments would also be effective for at least some other types of pathogens. In both of the ionisation and reduction steps, pathogen particles or viral aerosols can be inactivated or eliminated, and may be removed. Therefore, by virtue of having both these steps, sterilisation can be more effective than for systems using ionisation alone. The use of both ionisation and electrical potential reduction steps or stages can result in a significant or complete removal, inactivation or elimination of the infectious substances, without the use of UV lights, as have been shown in laboratory testing.

The processed airflow will be drawn out of the charge reduction zone 108 by the air pressure, where they continue to travel towards the outlet(s) 103. Further electrodes 109 which are negatively charged may optionally be provided within the housing 101 between the charge neutralisation zone 108 and the outlet(s) 103, to again ionise the air prior to its exit. The further electrodes 109 will generally be charged to a lower voltage than that generated at the ionising electrodes for initially ionising the aerosols and/or particles. They preferably will be charged to a level at which occurrence of static electricity will be minimised. Sensors such as ozone sensors 110 or ion sensors 111 may optionally be provided at or adjacent the outlet(s) 103 to detect the ozone or negative ion levels in the exiting air.

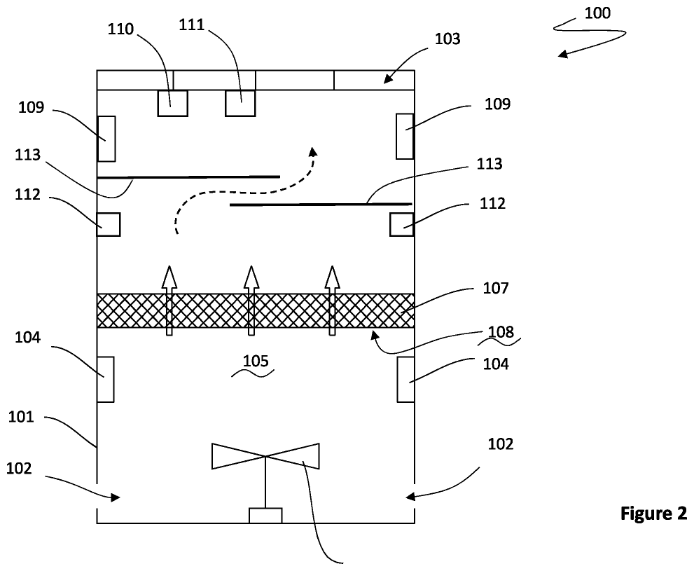

As shown in FIG. 2, the system 100 may optionally include one or more devices to provide internal sterilisation in the system 100. They work by destroying the infectious agents, whether those in an infectious aerosol or other pathogenic particles. They may be used to destroy infectious agents in the aerosol or infectious particles, that have been removed from the airflow by electrical attraction to the charge reduction stage 107.

One such device may provide one or more ultra-violet (e.g., UV-C) lights 112. If these lights are included, for safety, one or more covers 113 will also be provided to block the UV light from escaping and potentially creating a safety hazard. In this depicted embodiment, the covers 113 are arranged so as to also define a path for the air flow, as indicated by the dashed arrow. Such devices are not required for the purpose of removing, inactivating or eliminating infectious agents in the airflow. Another device that may be included is a heating module (not shown) intended to heat the system, or at least a portion thereof, so that it reaches a temperature high enough, and for a long enough duration of time to destroy the infectious agents. For instance, one or more heating elements may be provided to momentarily heat the system internally to 90° C. or above, but lower than a temperature which could interfere with the structure or function of the remainder of the system.

Another arrangement that may be provided is a chemical sterilisation arrangement, for applying a chemical to the charge reduction stage to chemically destroying the infectious agents.

The aforementioned devices, if included, may be kept on during the operation of the ionisation and charge reduction processes so as to destroy the infectious agents in real time. Alternatively or additionally they can be turned on at a time when the ionisation and charge reduction processes have been turned off.

The aforementioned option may also be provided in other embodiments disclosed in this specification.

In the embodiments disclosed in this document, the system 100 may optionally include a filter device 114 to trap larger particles in the air flow, such as dust. The filter device 114 may additionally trap smaller or fine particles. The selection of the filtration function can be chosen by the skilled person. One example is shown in FIG. 2, where the system 100 includes a filter device 114 located near the air inlets 102. In other embodiments, two or more filter devices 114 may be included. Multiple filter devices may be positioned at different parts within the housing 101. They may be configured to provide different filtration functions, e.g., to trap large particles, small particles, or fine particles, or aerosols, or a combination of two or more of these. The filter 114 is located adjacent the air inlet(s) 102 in FIG. 2. However, additionally or alternatively, the filter may be provided elsewhere. For instance, one or more filters can be provided inside the charge reduction zone 108. For instance, the charge reduction stage 107 may itself provide air filtration device to perform the dual function of air filtration and charge reduction. The charge reduction stage 107 may also be made of a material which is itself infused with an antiviral or antibacterial agent, such as silver.

In FIG. 1 and FIG. 2, the charge reduction zone 108 is defined by the physical space taken by the charge reduction stage 107. There may be two or more units of the charge reduction stage 107 in the system 100, to provide different zones of charge neutralisation. Charged (ionised) aerosols or particles that pass through a charge reduction stage 107 without contacting the surface of one charge reduction stage 107 may possibly come into contact with the next charge reduction stage 107

Further, the multiple units of the charge reduction stage 107 may have different electrical potentials. For example, a charge reduction or neuralisation device 107 which is provided later in the flow path (i.e., closer to the outlet 103) may be configured to have provided a larger electrical potential differential relative to the ionised aerosols and/or airborne particles, compared with the previous charge reduction stage 107. A stronger discharge current may thus be expected upon contact of the charged. Therefore, aerosols or airborne particles, that are missed by one charge reduction stage 107 or that are not inactivated by contact with the charge reduction stage 107, may be removed, eliminated, or inactivated by a later, possibly “stronger” charge reduction stage 107.

The embodiments shown in FIGS. 1 to 3 include an ionisation zone 105, followed by a charge reduction zone 108, and then the optional further ionisation zone near the outlet 103. In further embodiments, multiple ionisation zones 105 or multiple charge reduction zones 108, or both, may be included. Two or more of the multiple ionisation zones may be provided with different “strengths”—i.e., where the ionising electrodes are charged to different voltages. There is no requirement as to whether the different voltages should be increasing or decreasing, from the inlet(s) to the outlet(s). For instance, where the ionising electrodes are needle electrodes, the voltages at the electrode tips, in four ionisation zones from the inlets to the outlets, may be −5 KV (kilo-volts), −6.5 KV, −7 KV, −5 KV, or they may be −5 KV, −5.5 KV, −6 KV, −6.5 KV, or they may be −7 KV, −10 KV, −5 KV, −5 KV, or they may be −10 KV, −7 KV, −6 KV, −5 KV. Ionisation zones having other voltage levels, either in constant or changing relationships with each other, may be provided.

Two or more of the charge neutralisation zones may also be provided with different “strengths”, i.e., being provided to have different electrical potential. The ionisation zones 105 and the charge reduction zones 108 may alternate. One ionisation zone 105 may be followed by two or more charge neutralisation zones 108 in the flow path. A series of two or more ionisation zones 105 may be provided to precede a one or more charge reduction or neutralisation zones 108. There may be a gap between successive charge reduction or neutralisation zones 108 (e.g., see FIG. 9) by the placement of two successive charge neutralisation devices 108 spaced from each other. There may be a gap between two successive ionisation zones (see FIG. 8).

FIGS. 4 to 9 schematically depict different arrangements of charge reduction zones 108 and ionisation zone(s) 105, not necessarily to scale. These are provided as examples only, and are not exhaustive of the possibility of arrangements. For simplicity, in these figures only the different zones will be labelled, and the other components in the system such as the electrodes, optional sensors or lights, fan, etc, are not shown.

The described system 100 therefore makes use of different processes of adsorption of ions to create ionised aerosols and/or particles, followed by charge reduction effecting a change in the electrical potential in the ionised aerosols and/or particles or to neutralise the aerosols and/or particles, and in doing so causing a temporary current flow to remove, inactivate or eliminate infectious agents The described system 100 potentially further includes other filtration (e.g., by chemical bombardment or impregnation) functions or sterilisation functions.

By way of example, the following parameters may be used in the implementation of the described system. It will be appreciated that the exact parameters may be varied without departing from the spirit of invention. The variation may depend on factors such as the setting in which the system is used (e.g., clinical, domestic, laboratory, commercial), the size of the room or chamber in which air is being sanitized, the desired filtration rate, etc.

In one laboratory experimentation, at an air flow rate of 50 cubic meters per hour, an input of 18 volt was provided to a transformer to generate a −6.5 kV (kilo-volts) voltage at the ionising electrodes. The power required for the ionisation may also be expressed in terms of wattage. For example, in another laboratory experimentation, to process a flow rate of around 28.3 litres per minute, the total voltage generated by the electrodes are up to −8.0 kV, and the power of the generated ion is at between 2 to 6 Watts.

Preferably, the ionising electrodes will be charged so as to generate negative ions at a concentration of at least 1000 ions per cubic centimetre (cm 3). During testing, a concentration of 19 million negative ions per cubic centimetre had been measured. It would be expected that a concentration of say 1 to 100 million, or preferably 1 to 30 million ions per cubic centimetre, could be feasible as the operating ion concentration range. However the disclosed methodology does not rely on there being a particular concentration of ions, as long as there is a sufficient concentration to ionise a sufficient amount of the aerosol and/or airborne particles for the purpose of airflow sanitisation.

An example of a charge reduction stage 107 which was used during prototype testing was a wire mesh, made of stainless steel, having a mesh design of 50 holes per centimetre squared. However, as mentioned above, other types of devices may be used for the charge reduction stage 107.

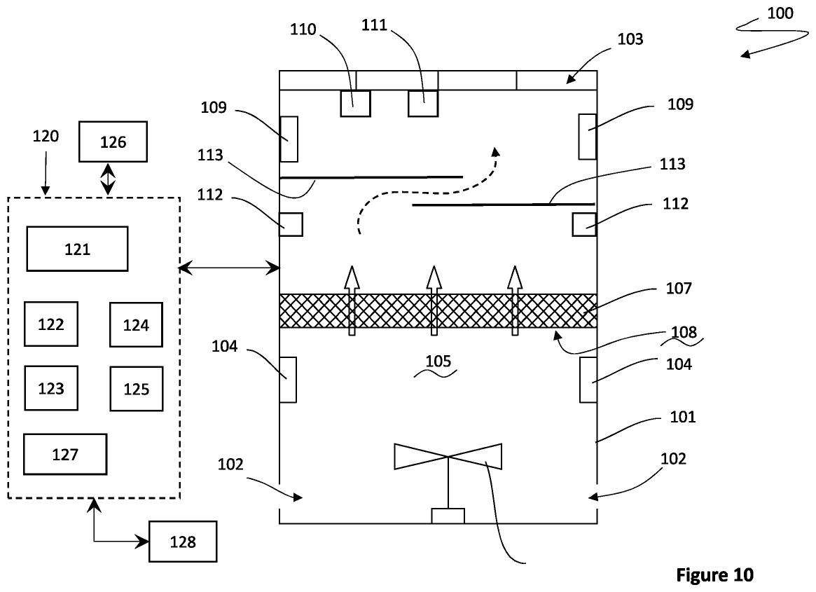

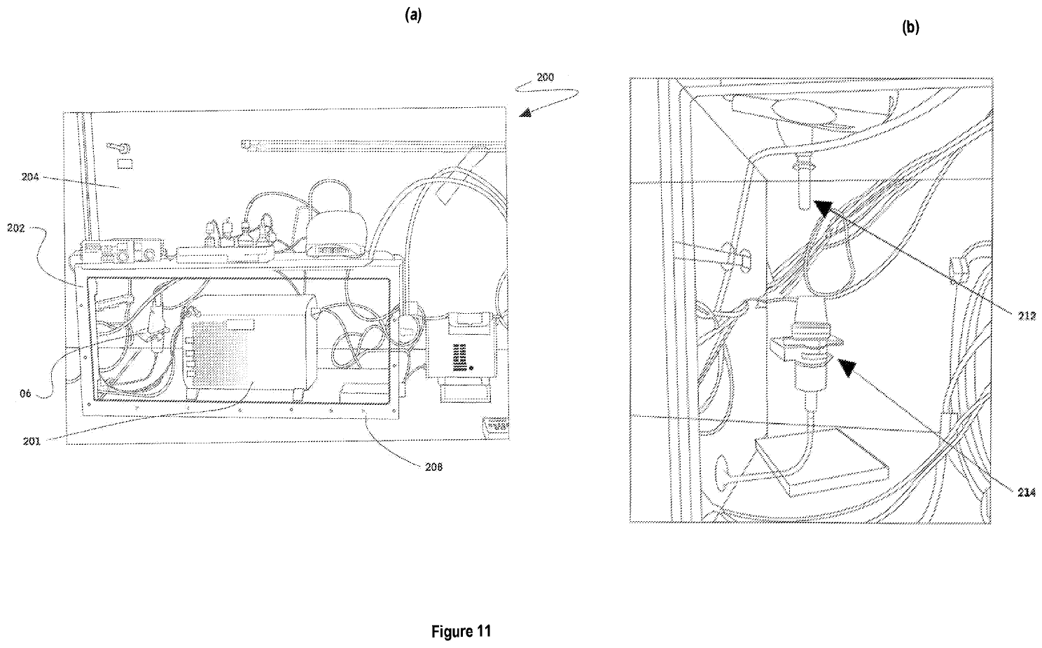

FIG. 10 schematically depicts the system 100 along with the control and power arrangements required to power the system 100. The system 100 depicted is that shown in FIG. 2, but it will be understood that a different embodiment of the system 100 can instead be included. The control and power arrangement may reside in a controller or in separate devices—the exact configuration may be dictated by space or design requirements. Here, the various modules are and shown within box 120, which conceptually represents the control and power arrangement. It is drawn in dashed lines to signify that the various modules contained therein do not need to be provided in the same physical device.

The control and power arrangement 120 includes a control module 121 which may include a processor or microprocessor, to control operation of the system 100. A user input/output (I/O) device 126, collocated with the control module 121 or coupled thereto using a wired or a wireless connection, may be provided to allow user monitoring or control of the operation of the system 100. The I/O device may be one unit, or may be separate input and output units. The control and power arrangement 120 may also include one or more communication module 127, to enable short range (e.g., Bluetooth®), near range, or long range (e.g., 3G, 4G, 5G, WiFi, etc) communication. The communication module 127, if included, enables the control and power arrangement 120 to send data transmission to and receive data transmission from, a mobile unit 128. The mobile unit 128 may be a remote control or a mobile device such as a smart device configured to communicate with the control and power arrangement 120 using compatible communications capabilities. This will allow the user to control the operation, such as turning the unit or particular components within the unit (e.g., decorative light, electrodes or reduction stages within particular ionisation or neutralisation zones, airflow) on or off, checking for any metric being monitored, adjust the operation setting such as voltage levels, the colours of any light included, airflow speed, etc.

To provide power supply for the operation of the system unit and various components therein, and for the control unit 120, the control and power arrangement 120 will further include a power supply arrangement. The power supply arrangement may include a mains power module 122 to receive mains power supply which is then converted to a direct current (DC) supply. The alternative current (AC) main power can be supplied via a USB (universal serial bus) connector and then converted to DC. There may be a DC power module 123 to receive DC power, such as from a battery. The direct DC supply or converted DC supply will be used to supply a voltage to the ionising electrodes 104, 109. Box 124 represents a negative voltage module which will supply to the required voltage or voltages so as to supply to the electrodes 104, 109. The negative module 124 may transform the input power supply to a different voltage suitable for supply to the ionising electrodes 104, 109, and potentially to the charge reduction stage 107 if the device of the stage is to be charged to a negative potential. It may also include the required circuitry, such as inverters to convert an incoming AC supply to a DC supply of the required polarity. Depending on the embodiments, there may alternatively or additionally a positive voltage module 125 which is configured to supply a positive potential to the ionising electrodes (if positive ions are used to ionise the aerosols and/or particles), and if applicable, one or more charge reduction stages 107. Both positive and negative voltage modules 124, 125 may be provided for greater operational or control flexibility.

In the above embodiments, the ionising electrodes will be configured to provide sufficient discharge to perform the function described, for the airflow to be sanitised. For example, in order to smoothly generate negative ions, in some embodiments, the ionising electrodes 104 (and optionally the further electrodes 109) may have a number of tips on the negative electrode configured to provide a point or two or more points where the charge density and the electric field strengths are concentrated. Typically, a larger tip curvature will result in a higher degree of concentration. i.e., the need to have a more pointed sharp site (typically, a large tip curvature, the charge density higher degree, high electric field strength). For example, the first negative electrode 105 and the second negative electrode 106 may be needle-shaped, and the tip corresponds to the needle-shape. The design may of course also be constrained by physical limitations imposed by the housing 101 or installation location. However, the specific design of the electrodes does not need to be otherwise limited. For example, for aesthetic reasons the electrodes may be configured to form a particular design, such as a star or another other shape.

As described in more detail below, prototype testing was done in separate experimentations, using a SARS-CoV-2 aerosol, and using a Getah virus aerosol, respectively. The experimentations were designed to detect the presence of infectious virus in the medium, when the test system was switched off, and compare the result with that obtained when the test system was switched on.

Experimentation Set Up, Methodology, and Result for System Testing with SARS-CoV-2 Aerosol

FIG. 11(a) is a picture of a system which was set up for testing in the laboratory, to ascertain the effectiveness of the described arrangement in sanitising an airflow containing airborne SARS-CoV-2 viral aerosol. The experimental setting 200 included a sealed Perspex box 202 that sat inside a biosafety cabinet (BSC) 204. The test unit 201 is placed inside the sealed box 202. Within the box 202 there was located a nebulizer 206 to create infectious aerosols and a slot to introduce and remove 96 well plates 208 containing medium and Vero E6 cells. The electronic controls on the exterior of the box allowed the nebulizer, the fan and the negative electrode in the test system to be switched on & off. A more detailed picture of the nebulizer is shown in FIG. 11(b), in which the container of virus solution 210 and a vent 212 for incoming air can be seen.

As described in more detail below, the testing was made to measure the viral concentration in the medium, when the test system was switched off, and compare the result with that obtained when the test system was switched on.

Negative electrodes off (no ionization) and no charge reduction—The nebulizer was loaded with 8 milliliters (ml) of SARS-CoV-2 (containing about 10⁶ log₁₀CCID₅₀/ml of virus) in a growth medium. For the growth medium, RPMI 1640 supplemented with 2% FCS (fetal calf serum) and buffered with 10 mM HEPES ((4-(2-hydroxyethyl)-1-piperazineethanesulfonic acid) was used. The box was sealed, and the nebulizer was run for 3 minutes while the Medair unit fan was switched on (powered to 3.5 volts). The nebulizer was then switched off and the fan run for a further 2 minutes. The lid on the 96 well plate was then lifted off and the fan was run for a further 15 mins. The fan was then switched off, and aerosols were vented by switching on an evacuation pump and by opening the vent above the nebulizer for 2 minutes. The 96 well plate in the box was exchanged via the slot in the box while the pump maintained a negative pressure with the vent above the nebulizer closed. The 96 well plate was recovered, a new sterile lid was placed on the plate, the plate was sprayed with 80% ethanol and was then transferred to the incubator in a sealed box and cultured for 6 days to evaluate viral cytopathic effects (CPE) which indicate the presence of infectious virus.

Negative electrodes on (ionisation on) and charge reduction on.

The above procedure was repeated with the negative electrodes on (18 volts), each time for a duration including 2 minutes of nebulization, 2 minutes with the lid on medium wells, 15 minutes with the lid off the medium wells, and 2 minutes of venting).

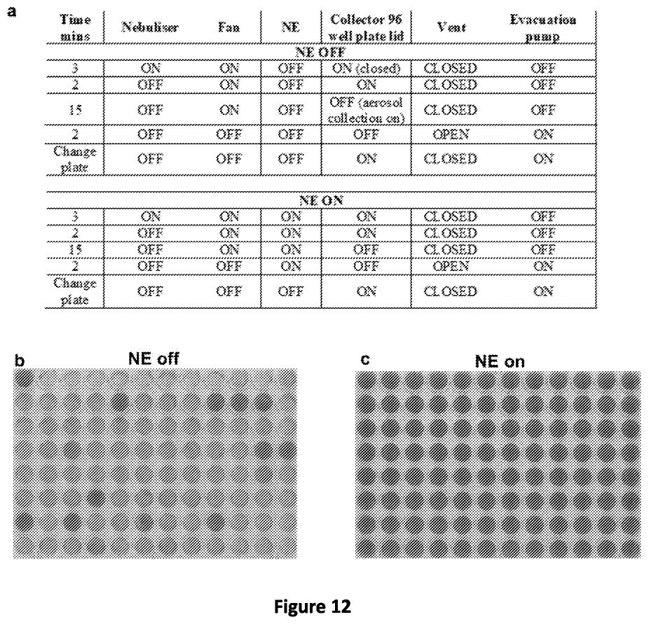

Results. In prototype testing as laid out above, an experiment under the aforementioned “negative electrodes on” configuration demonstrated a 100% removal of infectious aerosols, as compared with the amount of viral material detected in the wells of growth medium after the “negative electrodes off” experiment. FIG. 12(a) shows the switching sequence of the various components included in the system shown in the photograph of FIG. 11.

When the Negative Electrodes (NE) were switched off, 85 out of 96 wells captured infectious aerosols, with the wells of the 96 well plate with the Vero E6 cells containing 200 ul of medium supplemented with 5% FCS (fetal calf serum). Viral cytopathic effects (CPE) were indicated by the reduced intensity of blue/purple crystal violet staining in the well—see FIG. 12(b). When the Negative Electrodes (NE) was switched on, 0 out of 96 wells showed CPE, indicating that no infectious aerosols were detected in the wells, and that in this setting the device removed 100% of infectious aerosols—see FIG. 12(c).

In prototype testing as laid out above, an experiment under the aforementioned “negative electrodes on” configuration demonstrated a 100% removal of infectious aerosols, as compared with the amount of viral material detected in the wells of growth medium after the “negative electrodes off” experiment. FIG. 12(a) shows the switching sequence of the various components included in the system shown in the photograph of FIG. 11.

When the Negative Electrodes (NE) were switched off, 85 out of 96 wells captured infectious aerosols, with the wells of the 96 well plate with the Vero E6 cells containing 200 ul of medium supplemented with 5% FCS (fetal calf serum). Viral cytopathic effects (CPE) were indicated by the reduced intensity of blue/purple crystal violet staining in the well—see FIG. 12(b). When the Negative Electrodes (NE) was switched on, 0 out of 96 wells showed CPE, indicating that no infectious aerosols were detected in the wells, and that in this setting the device removed 100% of infectious aerosols—see FIG. 12(c).

Experimentation Set Up, Methodology, and Result for System Testing with a Getah Virus Aerosol

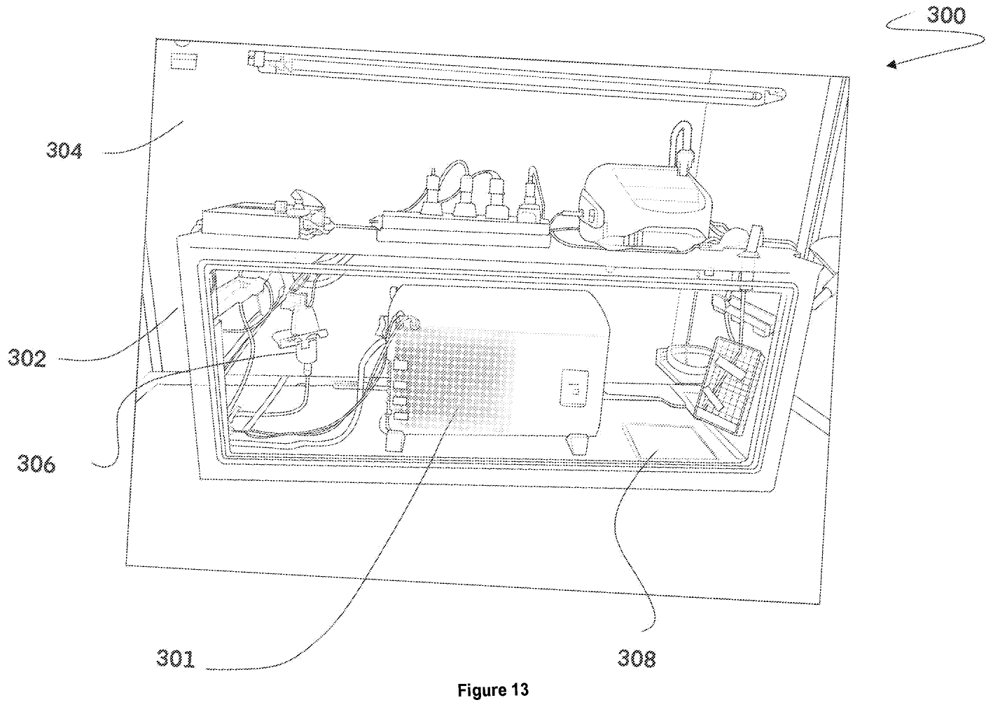

Set-up. FIG. 13 is a picture of a system which was set up for testing in the laboratory, to ascertain the effectiveness of the described arrangement in sanitising an airflow containing Getah virus viral aerosol.

The experimental setting 300 included a sealable Perspex box 302 that sat inside a biosafety cabinet (BSC) 304. Within the box 302 there was located a test unit 301 of a proposed system. Also located in the Perspex box 302 is a nebulizer 306 to create infectious aerosols, and a wet-surface aerosol collector 308, being a 96 well plate. The 96 well plate includes in each well 200 microliters of RPMI1640 medium with 5% FCS, and 10,000 Vero E6 cells plated the previous day.

The process to test the result in the wet collector is as described in the experimentation procedures below.

Negative electrodes off (no ionization) and no charge reduction. The nebulizer was loaded with 2 ml of GETV in RPMI 1640 supplemented with 2% FCS to which had been added 100 ul of GETV 10^{8.2} TCID50. The box was sealed and the nebulizer run for 2 minutes while the Medair unit (i.e., test unit) fan was switched on (at 3.5 volts). The nebulizer was then switched off and the fan run for a further 2 minutes. The lid on the wet collector was then lifted off and the fan run for a further 10 minutes. The fan was switched off, and then the UV switched on, and the aerosols were vented via ports to vacuum for 2 minutes. The BCS box was opened and the 96 well plate recovered, sprayed with 80% ethanol and cultured for 6 days to evaluate the viral cytopathic effects (CPE).

Negative electrodes on (ionisation on) and charge reduction on. The procedure outlined above was repeated with the negative electrodes on during the 2 minute nebulization period. The nebulizer was then switched off, and the test unit's fan was switched on for 2 minutes with the lid on wet collector, and for 10 minutes with the lid off the wet collector, followed by 2 minutes of venting (at 18 volts). At the end of the experiment the lid was replaced and the plate incubated for 6 days to assess the CPE.

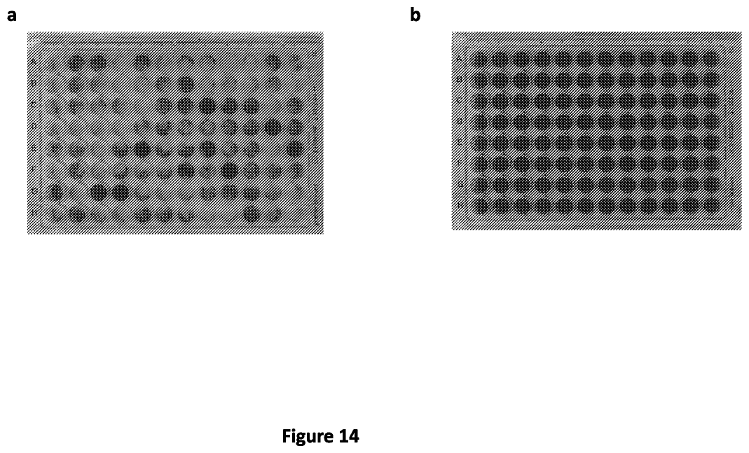

Results. The prototype testing showed clearly evident sterilizing and/or removal activity when the Negative Electrodes were on. Results from the wet collection showed 90 wells with CPE, indicating the presence of infectious virus after the run with the negative electrodes off (90 wells with light colours detected in FIG. 14a). After the run with the negative electrodes on, no CPE was detected in any wells (all dark coloured wells in FIG. 14b) indicating the absence of infectious virus.

Variations and modifications may be made to the parts previously described without departing from the spirit or ambit of the disclosure.

For example, if the system 100 were installed into or integrated with a system in which there will be an air flow (e.g., air circulation, filtration, or conditioning systems), the air purification or sanitisation system may be provided in the air path to act on the air drawn by the system, and a separate fan or other device to negative pressure may not be required. For instance, Figure X depicts a version of the system 100 shown in FIG. 1, which is installed in the airflow path 152 of an existing system 150, which includes its own airflow generation arrangement to enable the airflow 154.

The system 100 could be mounted in a duct 156 within the existing system as shown, so that the housing 101 provides an open inlet to accept incoming airflow, and an open outlet 103 via which exist airflow leaves the housing 101. Alternatively, the system could, by its housing 101 provide a duct section or a section of the structure in which the air flows. Alternatively, a duct section or section of the existing system 150 may be configured to function as the housing 101 of the system, the system components being provided therein to perform the required functions.

The system 100 could be attached to or integrated with a wearable device such as PPE, to process the air flow and provide the cleaned air for breathing by the wearer. In the wearable embodiments, the system 100 would need to have a built-in fan to drive the airflow in the PPE, or draw in ambient air to process it, or both. The system 100 would also be expected to include batteries to power the components.

As shown by the experimentation, the system described herein has been demonstrated to remove, inactivate, or eliminate 100% of the infectious aerosols in prototype testing. This departs from the current art where HEPA (high efficiency particulate air) filters are included in existing devices to capture the infectious aerosols. The system 100, compared with systems requiring HEPA filters, are much less costly to manufacture, requires less power to operate, and weighs less as the system 100 does not need as powerful (and thus heavy) a fan unit and does not require same amount of battery to run. The system 100 therefore provides significant technical advantage in comparison with existing arrangements utilising HEPA filters. This makes it possible for the system to be provided in a wearable device (e.g., PPE) or portable device.

It will be appreciated other variations of the system 100 may also be provided in an existing airflow path of another system, so as to be integrated therein. This allows the air purification or sanitisation system 100 to be retrofitted to existing building air circulation or air treatment systems.

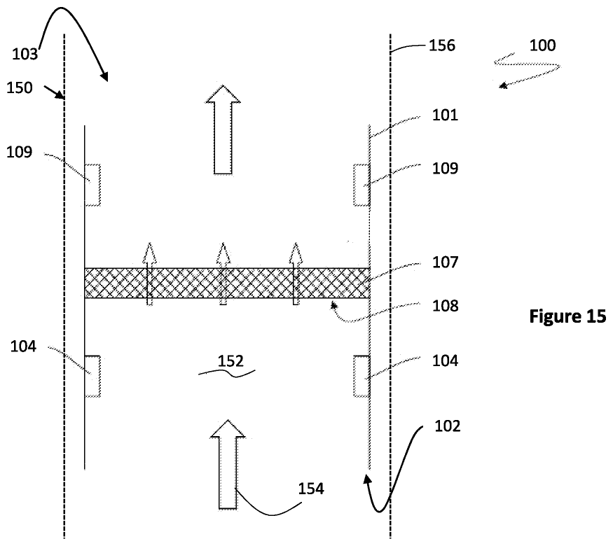

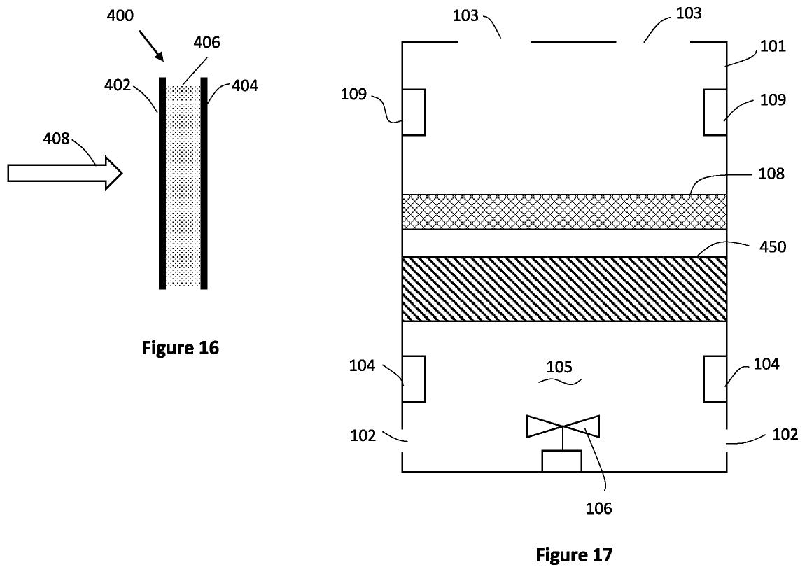

As alluded to earlier in the specification, embodiments of the system may also be provided one or more air filtration device 114 to provide an air filter function. Where multiple filtration devices are provided, they may be of different structure, material, or both. An example filtration device 400 is shown in FIG. 16. The filtration device 400 includes a layered construction comprising a first outer layer 402 and a second, opposite, outer layer 404. The first and second outer layers 402, 404 are spaced apart, there being a middle layer 406 in between. The layers 402, 404, 406 may utilise the same material, or they may be different. In a preferred embodiment, the outer layers 402, 404 are fabric layers comprising silver fibres. In a further preferred embodiment, the outer layers 402, 404 are fabric layers comprising silver fibres, and the middle layer is comprised of an activated carbon material. The device 400 is oriented such that it is located generally across the direction of the flow path 408 as defined by the housing 101 of the air purification or sanitisation system 100, at the location where the device 400 is placed. The air flow shown in FIGS. 1 to 10 and 15 is generally axial in the air path, where the air passes the ionisation or charge reduction stages. However, this is not essential. In any embodiment of the system, the system 100 may include an air redirection device which is configured to redirect the airflow so as to disturb the otherwise laminar airflow, particularly in the portion of the system between the ionising electrodes 104 and the charge reduction stage 107. The disturbed airflow will thus include flows in different directions and possibly different speeds. The device 550 is in this way considered to introduce or encourage turbulence or disturbance in the airflow. This helps to lengthen the time that it takes for the airflow to travel between the ionising electrodes 104 and the charge reduction stage 107. In other words, the air redirection device will be positioned between the ionisation zone 105 and the charge reduction zone 108.

As ions generated by the ionising electrodes 104 and the air are drawn through the air direction device, the degree or duration, or both, of the mixing between the ionic discharge and the incoming air, is increased, in comparison to embodiments where the air redirection device is not included. The increased mixing helps to increase the likelihood of attachment between the aerosol or other airborne substances and the ions, to promote the ionisation of the aerosol or other substances in the airflow. As an example, the air redirection device may be a Tesla air flow arrangement, but other arrangements may be used to lengthen the travel time for the air flow to pass through the system, between the electrodes 104 and the charge reduction stage 107, to promote the ionisation. For example, FIG. 17 schematically illustrates a variant of the embodiment shown in FIG. 1, the difference being that the system 100 as depicted in FIG. 17 further includes an air reduction device 450 located generally adjacent the ionising electrodes 104.

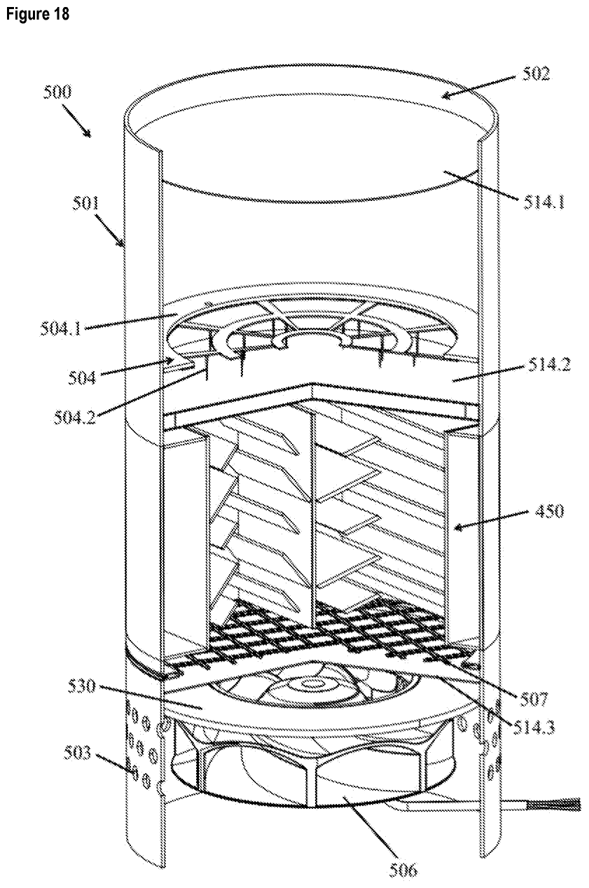

FIG. 18 is a cut-away view of an embodiment of the system including several purification or filtration devices and an air redirection device. As shown, the system 500 generally includes a housing 501. The air inlet 502 generally at or adjacent one end of the housing 501. As shown, a filter 514.1 is preferably provided adjacent the inlet 502. The filter 514.1 helps filtering out some larger particles in the air, such as dusts and debris. The filter 514.1 used in this embodiment is a fabric layer comprising silver fibres. The choice of the silver fibre helps to reduce the amount of odour particles in the airflow. Other materials could instead be used to provide the filter.

The depicted system 500 includes an ionisation device 504 configured to discharge ions into the airflow. The ionisation device 504 in this embodiment comprises a scaffold, or frame, 504.1 which carries a plurality of electrodes 504.2 for generating the discharge. The electrodes 504.2 are needle electrodes. The electrodes 504.2 are also preferably evenly spaced across the scaffold 504.1. Generally, voltages required to generate ionic discharge are at around 8000 volts or more, but the exact voltage may be varied. In other embodiments the ionisation device 504 can take another configuration or have another location (such as on the housing wall) as long as sufficient ions can be generated and discharged into the airflow. Ionisation device mentioned in respect of other embodiments described in the disclosure may also be used. For instance, the electrodes mentioned in respect of FIGS. 1 to 10 and 15 may be used in the ionisation device 504.

A second filter device 514.2 is provided adjacent the ionisation device 504, downstream from the ionisation device 504. The filter device 514.3 performs a further filtering of the air flow, for further dust or particle removal, odour removal, or ozone removal, or a combination of these functions. The filer device 514.2 may be the filtration device depicted in FIG. 16, having the activated carbon sandwiched between fabric layers comprising silver. The use of the activate carbon in the filter device 514.2 helps to reduce the amount of ozone, if any, generated by the ionisation electrodes 504.2. It also has the benefit as the material does not attract the ionic charges generated. The use of the silver material in the fabric helps to further eliminate odour from the airflow.

The air flow then continues and will be directed to pass through an air redirection device 550, located downstream of the ionisation device 504. In this example the air redirection device 500 is provided in the form of a Tesla air flow arrangement, but another arrangement to encourage the mixing between the airflow and the ions may be used instead.

The air flow which exits the air redirection device 550 will be drawn toward and pass through the charge reduction device 507. In this embodiment the ion discharge device 507 is in the form of a stainless steel mesh. In preferred embodiments, the mesh is sized at 50 or 60 apertures per cm². This sizing has been shown in laboratory testing to provide a workable balance between the power required to drive the airflow and for interacting with the ionised airflow to performing the charge reduction function. It will be appreciated that other charge reduction devices 507 may be used.

A filter device 514.3 is provided downstream of the charge reduction device 507, although it is not required in all embodiments. This filter device 514.3 helps to trap or block larger dust or other particles that remain in the air. The use of the fabric comprising silver fibres helps to further reduce any odour in the air but other materials can be used instead.

The charged reduced airflow continues to be drawn (or driven) toward the outlet of the housing 501. In this case, the charged reduced airflow will pass through a backdraft barrier 530. The backdraft barrier 503 helps to prevent back flow of the charge reduced air toward the charge reduction device 507 again, and to ensure that the air will exit the outlets 503. The backdraft barrier 530 is required in this embodiment because the airflow is arranged to exit via outlets 503 provided around the fan 506 in the wall of the housing 501. However, it will be understood that the backdraft barrier feature is not necessary in all embodiments, and its inclusion will depend on whether backdraft prevent is required.

As shown, the airflow is being drawn by an axial flow fan 506 located at the outlet end of the housing 501. Thus, the fan 506 needs to be powerful enough to draw the airflow into the air inlet 502 and through the various stages or devices included in the housing 501. Another factor to consider is the volume of the air being treated. A higher fan speed will be required for a larger space, as can be determined by the skill person. In other embodiments, the location of the fan 506 may be changed, for instance to be moved closer to the inlet end of the housing 501 instead, to drive the airflow. It will be understood the fan 506 may be omitted if the system 500 is mounted within an airflow system with a driven airflow.

The air outlet, in the form of a plurality of outlet apertures 503, are provided on the wall of the housing 501, in the portion between the outlet end of the housing 501 and the fan 506. In alternative embodiments, the air outlet 503 may instead or additionally be provided to provide one or more openings formed in the outlet end of the housing rather than on the wall of the housing.

Thus, the system 500 shown in FIG. 18 can generally be considered to perform six action stages, being the filtration performed by the filter device 514.1, the generation of the ions by the ionisation device 504, the further filtration performed by the filter device 514.2, the turbulence introduction by the air redirection device 550, the charge reduction by the charge reduction device 507, and the third filtration by the filter device 514.3.

However, variants of the embodiment depicted in in FIG. 18, which may not necessarily perform all six action stages, are also intended to be encompassed in the scope of this disclosure. Some of these variants are already discussed in relation to potential alternative arrangements, e.g., for the air redirection, charge reduction, or ionisation. Further, the system 500 may include only one filter device, and the one filter device is not restricted to being located at any particular location as long as it filters the air prior to the air exiting the system. However it is beneficial to have the filter located nearer the inlet end. Also, the filters may utilise alternative constructions or materials.

In the above embodiments, the system is generally depicted as comprising a housing that provides a straight channel in which the air flows. However this channel, or indeed the housing itself, is not restricted to being straight or linear. Rather, the housing may be provided in a form, shape, or configuration, which defines a curved flow path, or a flow path including one or more turns. This may be useful particularly in situations where the housing needs to be designed to fit into an existing system or be designed with a space constraint.

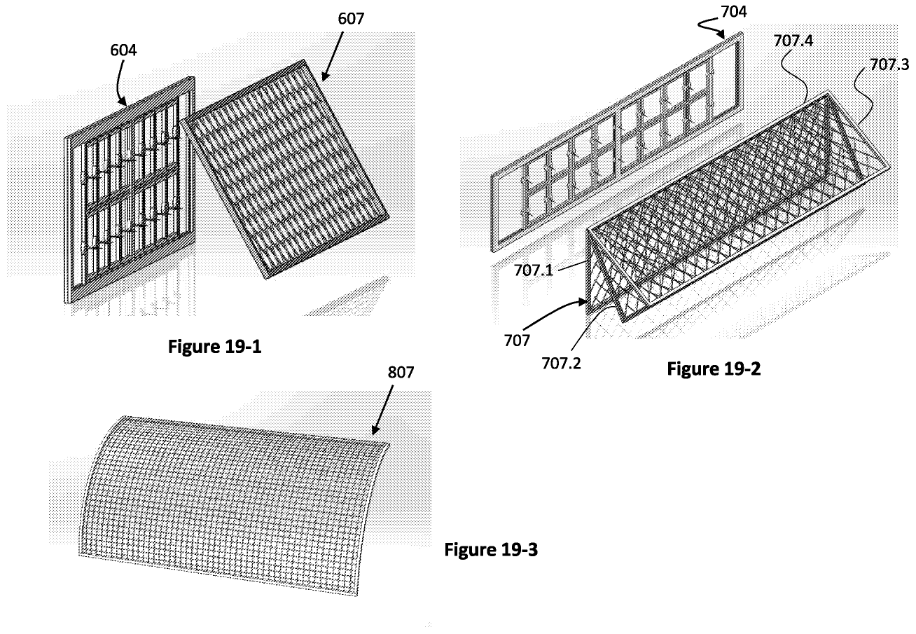

The various stages or devices included in the system 100, 500, do not necessarily need to be parallel to each other. For example, the housing channel defining the flow path may have a tangential direction which changes. Thus, where there are components which are provided across the flow channel at a curved portion of the flow channel, it will be understood that they are provided generally across a tangential direction at that portion of the flow channel. For instance, as seen in FIG. 19-1 the ionisation device 604 is located at an angle to the charge reduction device 607.

In the embodiments, each charge reduction device 107, 507 optionally can comprise two or more components or parts. For example, as shown in FIG. 19-2, the charge reduction arrangement 707 includes three portions 707.1, 707.2, 707.3, which are positioned at different angles. The portions 707.1, 707.2, 707.3 may be generally collocated along one edge 707.4. The edge 707.4 may be a spine where the portions 707.1, 707.2, 707.3 are attached or connected to each other, and from which the portions 707.1, 707.2, 707.3 fan out. Alternatively the portions may be separately mounted in the housing for the system where they are used, but arranged at the progressively changing angles. This charge reduction arrangement 707 may be useful in circumstances where there is a bend or curve in the flow channel and thereby the flow path through the charge reduction zone. The progressively changing angles of the portions 707.1, 707.2, 707.3 may generally follow the curve in the flow channel.

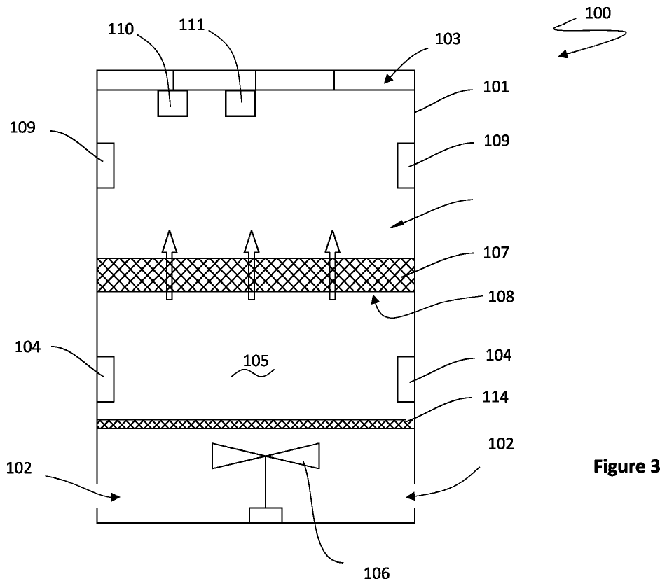

Further, although the various stages in the system, particularly some embodiments of the ionisation device and of the charge reduction device, have been illustrated as being generally planar, this is not a requirement for all embodiments. These devices can assume a curved configuration, for example, to fit any space or physical constraints imposed by the location of installation or the desired form factor for the system. For instance, in FIG. 19-3, the charge reduction device 807 has a curved rather than a planar configuration like the devices 607, 707 shown in FIGS. 19-1 and 19-2.

In the above embodiments, in the air ionisation zone, the aerosols and/or airborne particles are ionised with a negative charge—e.g., via a discharge of electrons from the electrodes—and a current flow is induced to flow from at least some of the ionised aerosols and/or particles toward a conductive device of the charge reduction stage 107 which provides a reduction or neutralization of the negative charges. However, in alternative embodiments, the aerosols and/or particles may be ionised with a positive charge, by applying a positive potential to the electrodes, and the charge reduction or neutralisation device 107 will be configured to reduce or neutralise the positive charges.

In the above embodiments, provided they fulfil the required functions, the electrodes may be formed of different materials such as gold, silver, platinum, or carbon fibre. They may be a metallic or non-metallic material plated with gold, silver platinum or copper. They may also take various shapes, such as needle, circular, or may have attractive designs that enhance the aesthetic of the system, particularly if the electrodes will be visible.

Other components may be provided in the housing to generate additional functions—such as non-UV lights which provide a visible light through the housing, so that the unit can function as a lamp or lighting device, or to improve the aesthetics of the unit.

In the embodiments where the system includes the air pressure generation to create the air flow, the air pressure may be provided by a fan or an air pump to generate a positive pressure, or it may be provided by a vacuum to generate a negative air pressure.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. An air purification system, comprising:
an air inlet for accepting an airflow into the system and an air outlet for the airflow to exit the system, with a conduit between the air inlet and the air outlet,
disposed within the conduit in the direction of air flow, the system further includes:
at least one ionizer creating an air ionisation zone within which, in use, aerosols and/or airborne particles in incoming air are ionised become charged, and
a charge reduction stage creating a first charge reduction zone, the charge reduction stage comprising a charge reduction device which is located across a flow path of the airflow, terminating the air ionisation zone, such that the charged aerosols and/or particles pass therethrough, and in passing through the charge reduction stage the charged aerosols and/or particles are attracted to the charge reduction stage and on contact with the charge reduction stage become at least partially discharged, the action of charging and subsequent discharging biologically inactivating at least some aerosols and airborne particles.

2. The system of claim 1, wherein the ionizer comprises a plurality of electrodes distributed within the air ionization zone and charged, in use, to generate a negative ion discharge so as to ionise aerosols and/airborne particles in incoming air passing through the air ionisation zone.

3. The system of claim 2 wherein the electrodes are charged to provide a corona discharge.

4. (canceled)

5. (canceled)

6. The system of according to claim 2, wherein the ionizer comprises a frame onto which the electrodes are mounted, the frame being generally positioned across the flow path.

7. (canceled)

8. The system according to claim 1, comprising one or more further ionisation zones.

9. The system according to claim 5, wherein at least two of the two or more ionisation zones are configured to charge the particles in the incoming air to different electrical potentials.

10. The system according to claim 6, wherein the ionisation zone which is closest to the inlet is configured to charge the aerosols and/airborne particles in the incoming air to provide a least ionisation level compared to other ionisation zone(s).

11. The system according to claim 1, further comprising one or more further charge reduction stages.

12. The system according to claim 8, wherein the ionizer comprises a frame onto which the electrodes are mounted, the frame being generally positioned across the flow path, and wherein at least two of the charge reduction stages and two of the ionisation zones in the conduit are provided in alternating fashion.

13. (canceled)

14. (canceled)

15. The system according to claim 1, wherein 1) the charge reduction device comprises a structure having a textured surface, or 2) a material of the charge reduction device comprises an anti-bacterial or sterilising additive or both.

16. The system according to claim 1, wherein the charge reduction device includes at least one mesh layer.

17. (canceled)

18. (canceled)

19. The system of according to claim 1, comprising at least two charge reduction devices arranged in series in the conduit, the two charge reduction devices having different electrical potentials.

20. The system according to claim 1, further comprising at least an exit ionisation zone which is provided adjacent the outlet.

21. (canceled)

22. (canceled)

23. The system according to claim 1, comprising an air redirection device configured to disturb the airflow as it travels from the ionizer to the charge reduction stage.

24. The system according to claim 14, wherein one or more of the following applies in relation to the air redirection device: 1) the disturbed airflow comprises flows in different directions, 2) the air redirection device is configured to define one or more airflow path therein which are longer than a length between the ionizer and the charge reduction stage, 3) the air redirection device comprises a Tesla air flow arrangement.

25. (canceled)

26. (canceled)

27. The system according to claim 1, comprising a filtration device located adjacent the ionizer.

28. (canceled)

29. (canceled)

29. The system according to 16, wherein the filtration device comprises a layered structure.

31. The system according to claim 17, wherein the layered structure includes outer layers which are fabric layers comprising silver fibres.

32. (canceled)

33. (canceled)

34. The system according to claim 1, retrofitted in an existing building, transport or vehicular air circulation system.

35. (canceled)

36. A method of air purification, including:
creating an air pressure to draw an airflow through a flow path;
providing positive or negative ions in the flow path, so as to ionise aerosols, airborne particles, or both in the flow path; and
altering an electrical potential of at least some of the ionised aerosols, airborne particles, or both, to biologically inactivate said at least some of the ionized aerosols and/or airborne particles, by placing in the flow path a charge reduction device comprising a charge reduction device which is located across a flow path of the airflow such that the airflow will pass therethrough with charged aerosols and/or particles passing therethrough after being ionised, and said at least some of the ionised aerosols, airborne particles, or both, will contact the charge reduction device and as a result of contact alter electrical potential to thereby biologically inactivating the ionised aerosols, airborne particles or both.

37.-38. (canceled)
